(19) United States
(12) Patent Application Publication
(10) Pub. No.: US 2012/0130392 A1
(43) Pub. Date: May 24, 2012

(54) NECROSECTOMY ASSIST DEVICE

(75) Inventors: Michael J. Levy, Rochester, MN (US); Todd H. Baron, SR., Oronoco, MN (US)

(73) Assignee: Mayo Foundation for Medical Education and Research, Rochester, MN (US)

(21) Appl. No.: 13/296,655

(22) Filed: Nov. 15, 2011

Related U.S. Application Data
(60) Provisional application No. 61/415,132, filed on Nov. 18, 2010.

(51) Int. Cl. A61B 17/26

(52) U.S. Cl. 606/114

(57) ABSTRACT

Endoscopic approach necrosectomy assist devices and methods of use to collect necrotic pancreatic tissue. One embodiment of the device includes a body, an expandable cap on a distal end of the body, an actuating mechanism for causing the cap to move between a reduced diameter retracted state and an expanded diameter operational state, a tissue collecting band on the cap, and a band actuating mechanism. Another embodiment of the invention includes a handle and a scoop on the handle. The scoop is movable between a reduced diameter retracted state and an expanded diameter operational state. The scoop is formed from a shape memory material and is self biased to the operational state. Blades, rake tips, cutters or abrasive can be included on the scoop.

### CROSS-REFERENCE TO RELATED APPLICATION

This application claims the benefit of U.S. Provisional Patent Application No. 61/415,132 filed Nov. 18, 2010, which is incorporated herein by reference in its entirety and for all purposes.

### TECHNICAL FIELD

The invention relates generally to a surgical instrument and a surgical method performed with the instrument. In particular, the invention is a necrosectomy assist device and a minimally invasive, endoscopic approach surgical procedure using the necrosectomy assist device to treat necrotic pancreatic tissue.

### BACKGROUND OF THE INVENTION

Patients suffering from pancreatitis can develop walled off pancreatic necrosis (WOPN). WOPN is the collection of liquid and solid necrotic debris in the pancreas. Surgical, percutaneous and endoscopic approaches are known therapeutic options for treating WOPN. Although endoscopic approaches are favored in some patients, these procedures can be time intensive. Necrotic debris can, for example, stick to the wall of peritoneal structures as a viscous material during these procedures, and be difficult to remove. There remains, therefore, a need for improved surgical instruments and procedures. In particular, there is a need for improved endoscopic instruments and procedures for treating WOPN.

### SUMMARY OF THE INVENTION

The invention is an improved necrosectomy device and method for using the device to collect necrotic pancreatic tissue. One embodiment of the device includes a body, an expandable cap on a distal end of the body, and a tissue collecting band on the cap. Another embodiment of this device includes an actuating mechanism for causing the expandable cap to move between a reduced diameter retracted state and an expanded diameter operational state. A band actuating mechanism is coupled to the band in yet another embodiment.

Another embodiment of the invention includes a handle and a scoop on the handle. The scoop is movable between a reduced diameter retracted state and an expanded diameter operational state. The scoop is formed from a shape memory material and is self biased to the operational state in still another embodiment of this invention. Blades, rake tips, cutters or abrasive can be included on the scoop.

### BRIEF DESCRIPTION OF THE DRAWINGS

FIG. 1 is a partially cross-sectioned illustration of a banding necrosectomy assist device in accordance with one embodiment of the invention.

FIG. 2 is a detailed sectional side view of the distal end of the device shown in FIG. 1 with the cap in a closed, retracted state.

FIG. 3 is a detailed sectional side view of the distal end of the device shown in FIG. 1 with the cap in an open, operational state.

FIG. 4 is an illustration of a surgical procedure including use of the necrosectomy assist device shown in FIG. 1 to treat walled off pancreatic necrosis.

FIGS. 5A, 5B and 5C are top, side and end views, respectively, of a portion of the handle and tip of a scoop necrosectomy assist device in accordance with another embodiment of the present invention in a closed, retracted state.

FIGS. 6A, 6B and 6C are top, side and end views, respectively, of a portion of the handle and tip of the scoop necrosectomy assist device shown in FIGS. 5A-5C, in an open, operational state.

FIG. 7 is an illustration of a surgical procedure on a pancreas using the device shown in FIGS. 5A-5C.

FIGS. 8A and 8B are top and side views, respectively, of a scoop necrosectomy assist device in accordance with another embodiment of the invention having a rake tip.

FIGS. 9A and 9B are top and side views, respectively, of a scoop necrosectomy assist device in accordance with another embodiment of the invention having a bowed tip.

FIG. 10 is an illustration of a balloon necrosectomy assist device in accordance with another embodiment of the invention.

FIGS. 11A and 11B are top and side views, respectively, of a scoop necrosectomy assist device in accordance with an embodiment of the present invention in an uncoiled state.

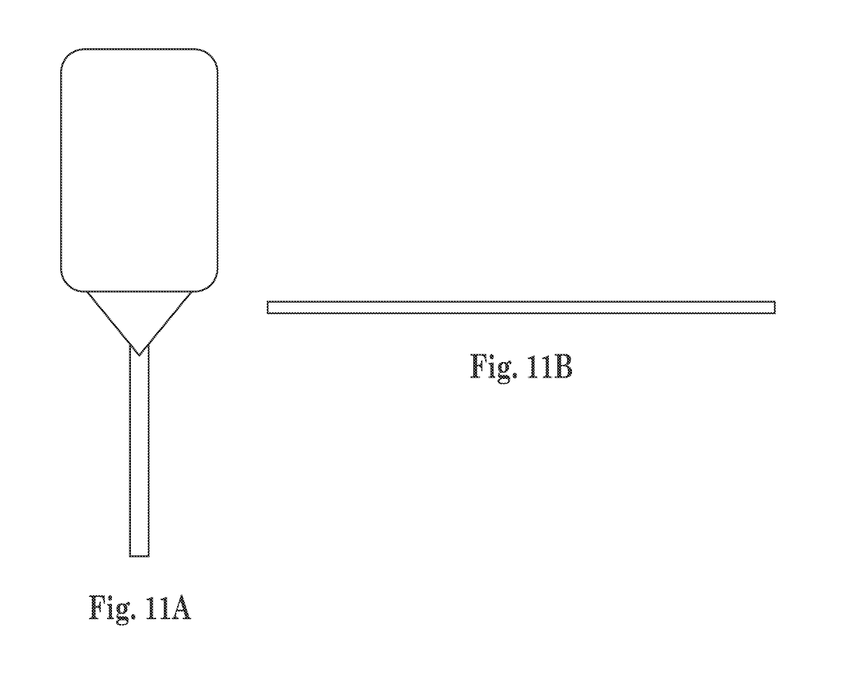

FIGS. 12A and 12B are top and side views, respectively, of the necrosectomy assist device shown in FIGS. 11A and 11B in a coiled state.

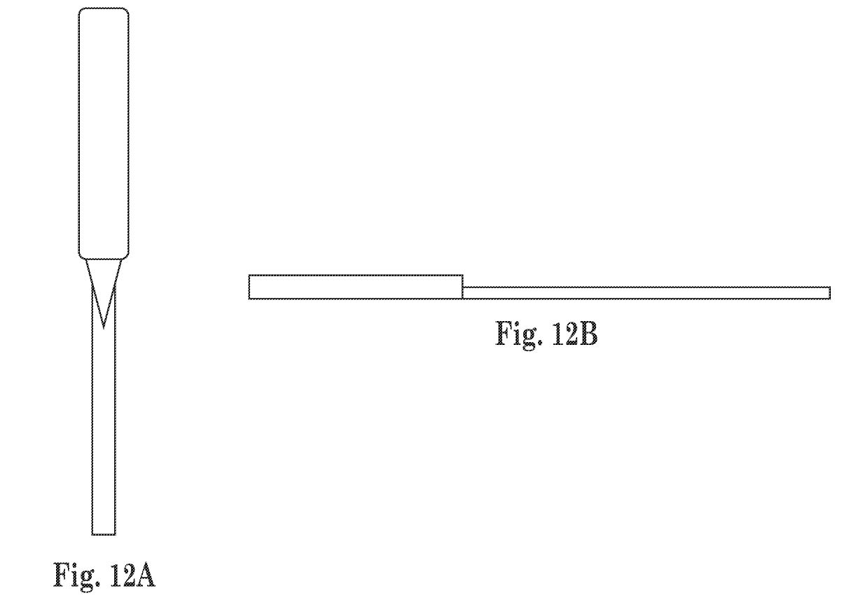

FIGS. 13-15 are top views of “cheese cutter” or razor blade necrosectomy assist devices in accordance with additional embodiments of the invention in an uncoiled state.

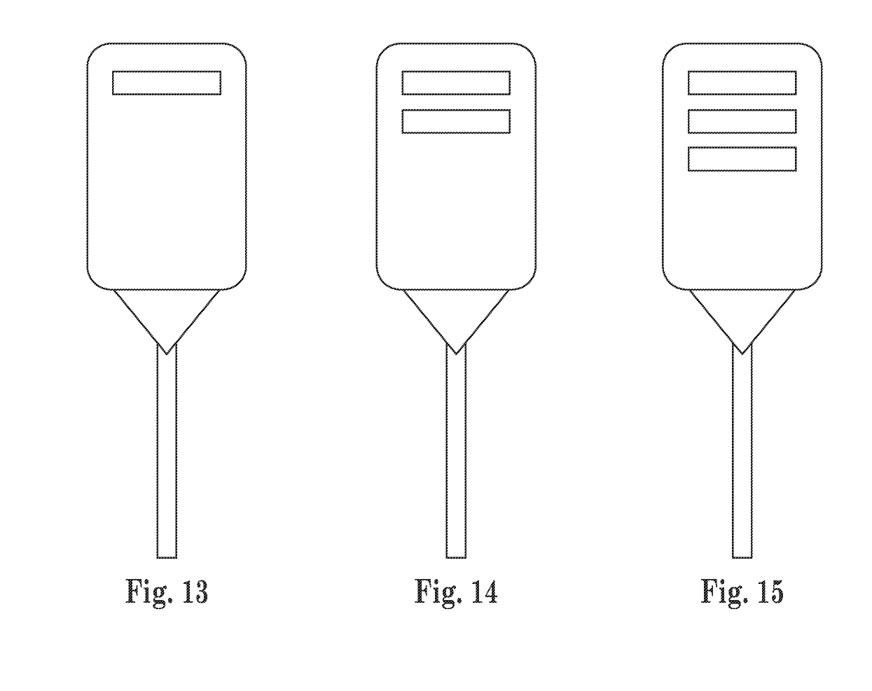

FIGS. 16A and 16B are top and side views, respectively, of a razor blade necrosectomy assist device in accordance with an embodiment of the invention in an uncoiled state.

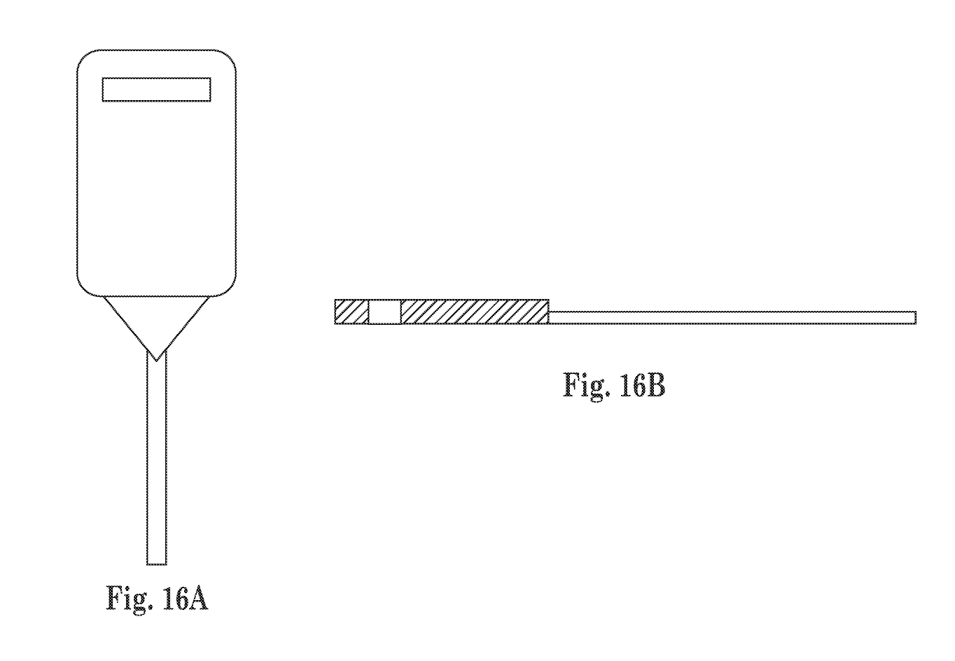

FIGS. 17A and 17B are top and side views, respectively, of a rake tip necrosectomy assist device in accordance with an embodiment of the invention in an uncoiled state.

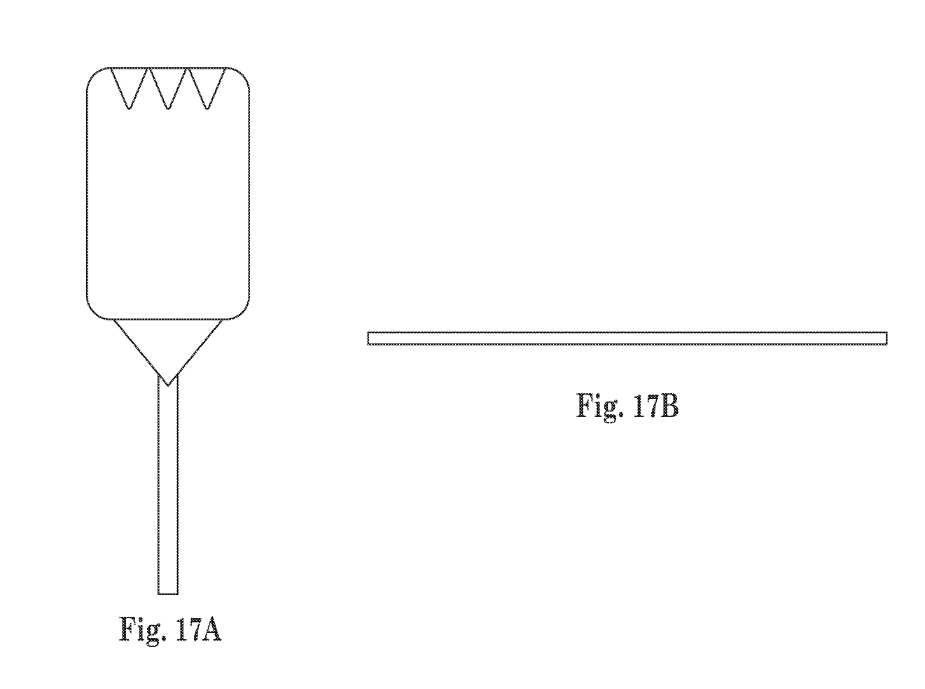

FIGS. 18A and 18B are top and side views of a “cigar cutter” necrosectomy assist device in accordance with an embodiment of the invention in an uncoiled state at open and closed cutting states, respectively.

FIGS. 19A and 19B are top and side views, respectively, of a necrosectomy assist device having water infusion ports in accordance with an embodiment of the invention in an uncoiled state.

FIGS. 20A and 20B are top and side views, respectively, of a bowed scoop necrosectomy assist device in accordance with an embodiment of the invention in an uncoiled state.

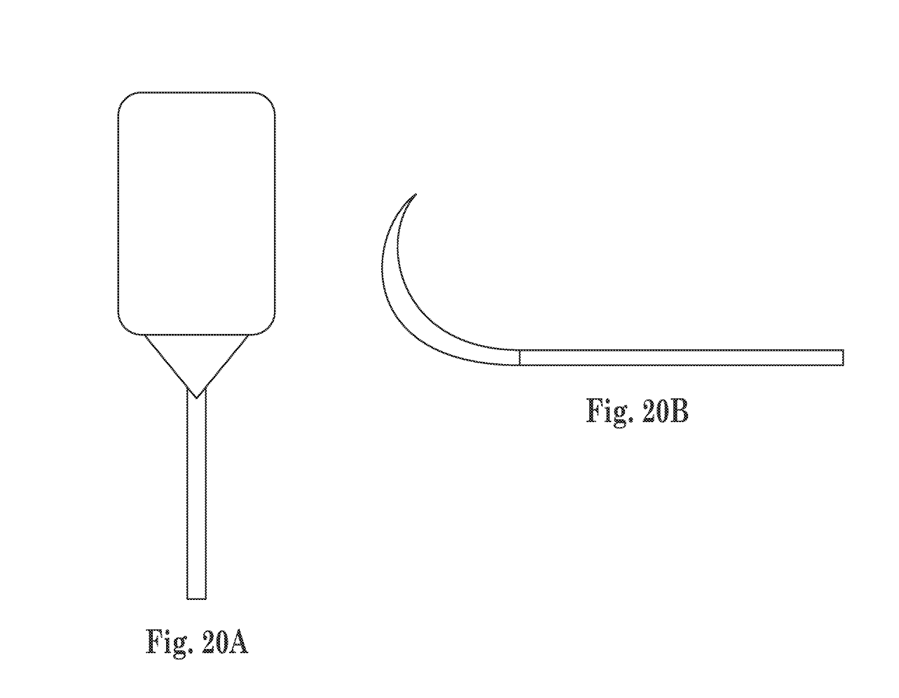

FIG. 21 is a top view of a suction and bander necrosectomy assist device in accordance with an embodiment of the invention.

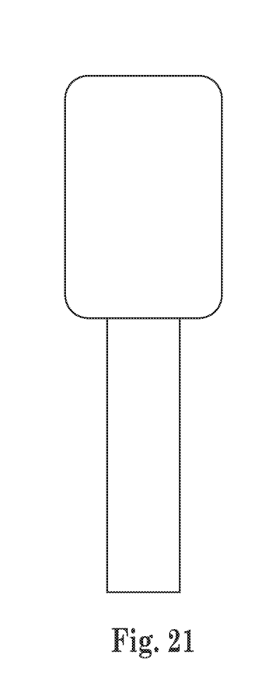

FIG. 22 is a top view of a suction and scoop necrosectomy assist device with back angled inserts in accordance with an embodiment of the invention in an uncoiled state.

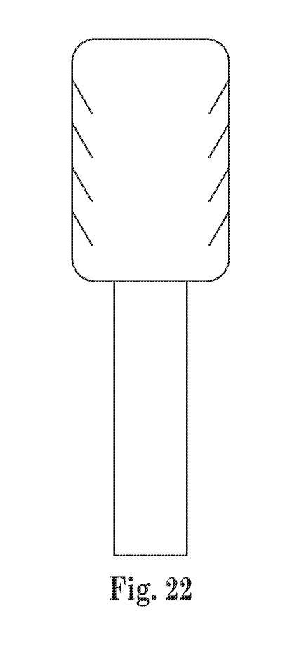

FIGS. 23A, 23B and 23C are top views of an adjustable suction and bander necrosectomy assist device in accordance with an embodiment of the invention in an uncoiled state.

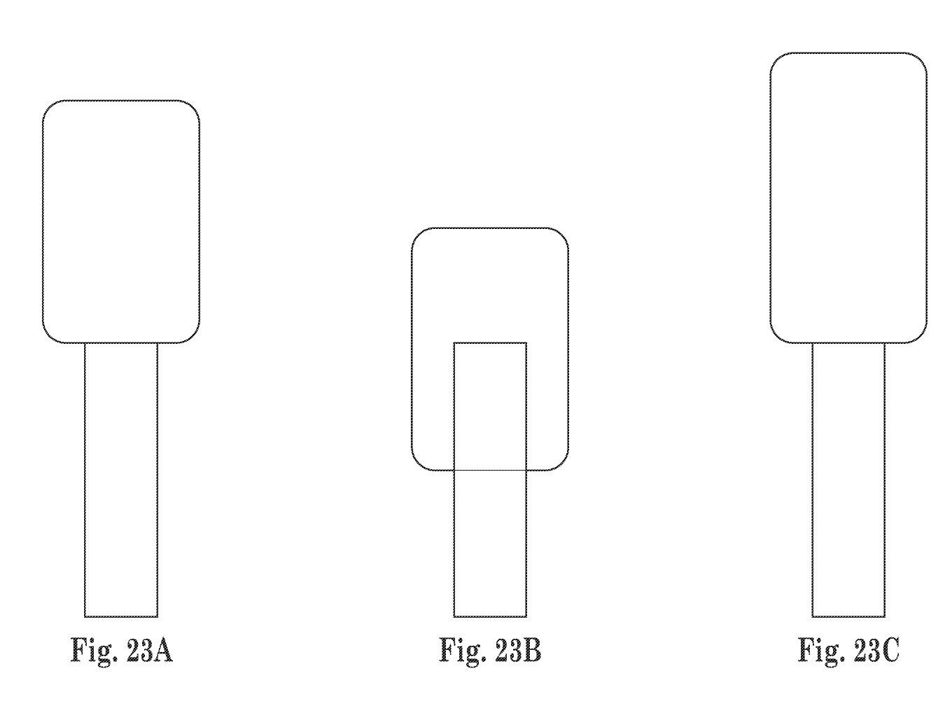

FIGS. 24A, 24B and 24C are views of an abrasive balloon necrosectomy assist device in accordance with an embodiment of the invention in different states of inflation.

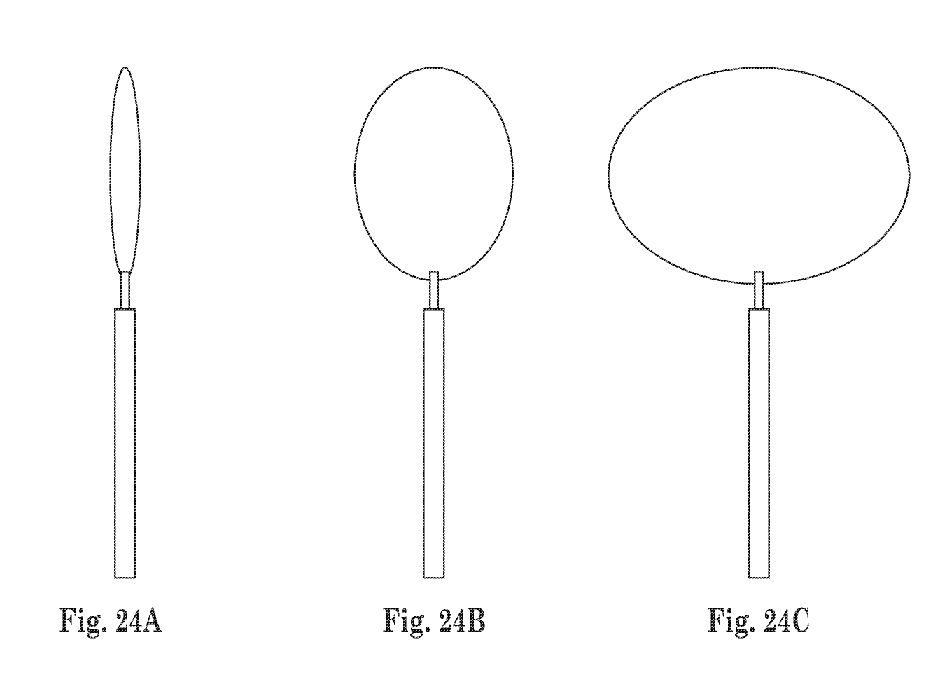

FIG. 25 is a detailed view of the balloon necrosectomy assist device shown in FIGS. 24A-24C.

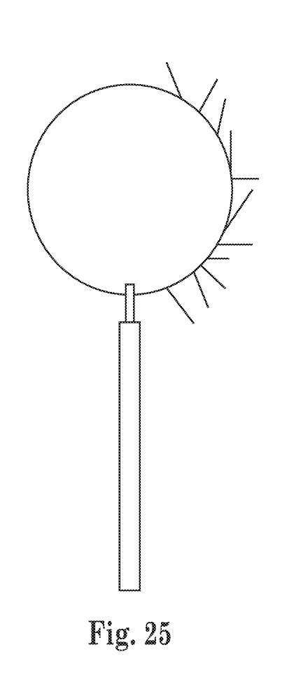

### DETAILED DESCRIPTION

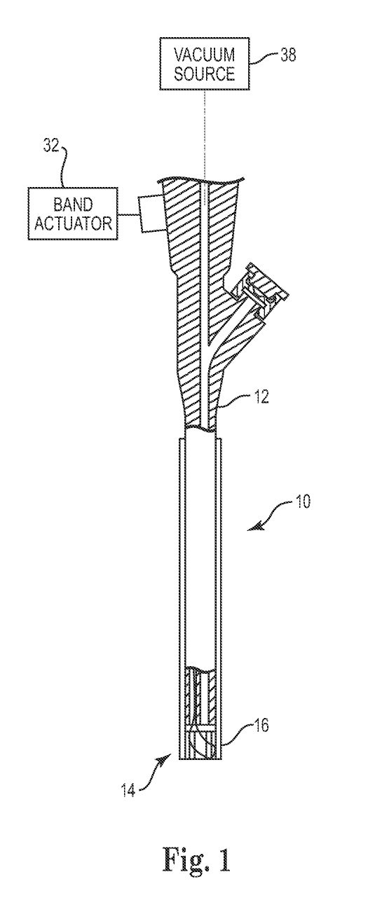

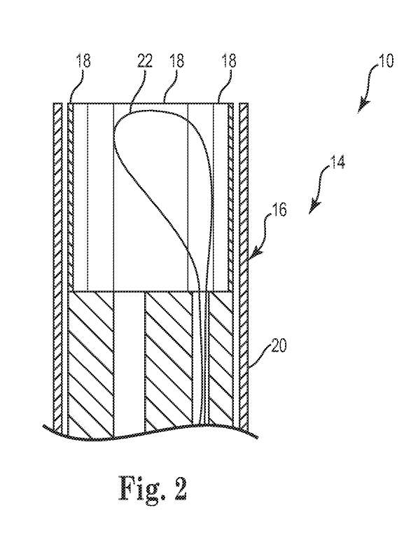

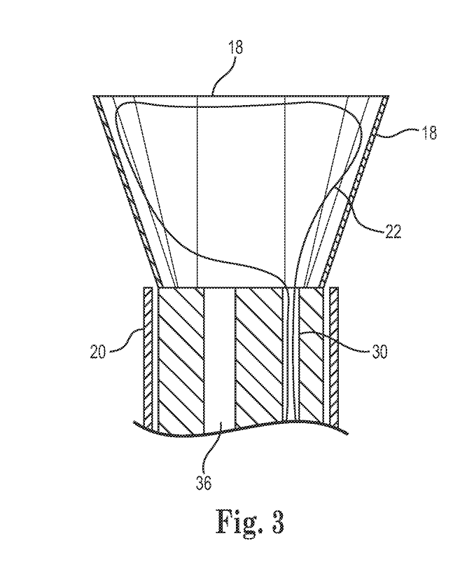

FIGS. 1-3 are illustrations of an banding necrosectomy assist device 10 in accordance with one embodiment of the invention. As shown, device 10 has a body 12 with a tissue collection tool 14. As perhaps best shown in FIGS. 2 and 3, tool 14 includes a cap 16 on the distal end of the body 12 that forms a chamber for collecting necrotic pancreatic tissue, an actuating mechanism 20 to move the cap between delivery and operational states, and a collection band 22. In response to actuation of the actuating mechanism 20, the cap 16 can be moved between the retracted delivery state shown in FIG. 2 and the open operational state shown in FIG. 3. The illustrated embodiment of cap 16 includes a plurality of overlapping and circumferentially-positioned leaf-type elements 18. Elements 18 are formed of elastic shape memory material such stainless steel, nitinol or polymers and are biased to an open, expanded diameter configuration shown in FIG. 3 to increase the volume of the chamber when the cap 16 is in the operational state. The actuating mechanism 20, which is shown as a tube or sheath, can be moved in a distal direction with respect to the body 12 and over the elements 18 to force the elements together against the elastic bias into a reduced diameter configuration and place the cap in the retracted delivery state.

Band 22 extends around a circumference of the cap 16 adjacent to the distal end of the cap. In the embodiment shown the band 22 is a snare type element formed from a length of elongated flexible material. Ends of this embodiment of the band 22 extend from the cap 16 through a lumen 30 in the body 12 and are connected to a band actuator 32. The body 12 also has a lumen 36 that extends into the chamber of the cap 16 and is configured to be connected to a vacuum source 38.

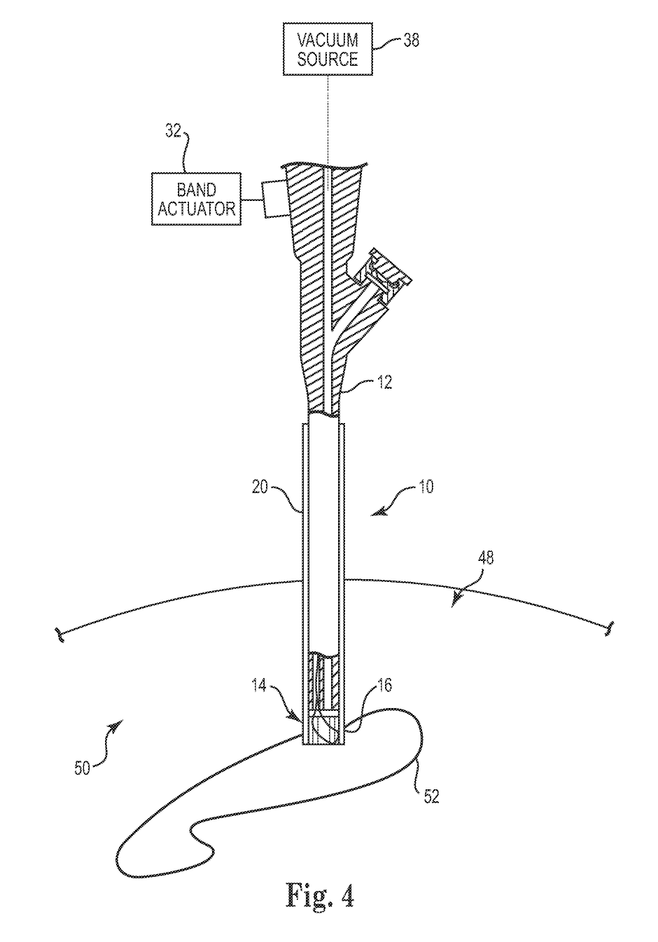

FIG. 4 is an illustration of a patient 48 undergoing a therapeutic surgical procedure during which the device 10 is used to treat walled off pancreatic necrosis (WOPN). As shown, the device 10 is inserted into the peritoneal cavity 50 through an endoscopic approach, and the cap 16 is positioned adjacent to the pancreas 52. The device 10 is inserted with the cap 16 in the retracted state. After insertion, the actuating mechanism 20 is actuated by the surgeon to cause the cap 16 to diametrically expand to its open and operational state. Suction can be applied to the cap 16 through the use of vacuum source 38 to draw fluid and tissue into the cap. Band actuator 32 can be actuated to resect the aspirated necrotic tissue. After the necrotic tissue is collected from pancreas 52, the actuating mechanism 20 can be actuated to move the cap 16 back to the retracted state, and the device 10 and collected tissue is removed from the patient.

The necrosectomy assist device 10 in accordance with the invention can take other embodiments. For example, the expandable and retractable cap can be formed from other structures. The actuating mechanism for the cap can take other forms. For example, a mechanical linkage can be coupled to the structure of the cap, and mechanically drive the cap between the open and closed states. The band and band actuator can also be embodied in different structures. For example, the band can be a rubber band structure, and the actuator a mechanism a structure for releasing the expanded band around tissue in the cap. The released band can then take a reduced diameter state and engage the collected tissue. The cap can be provided in multiple sizes and/or the degree to which the size of the cap can be expanded can be adjusted or vary from cap to cap. The device can then be adjusted or selected based on the amount of necrosis to be removed. Other features can also be added to the device. Examples include a cautery tip, prongs for removing material when the device is torqued, and back-angulated inserts along the inner wall of the cap to hold the collected tissue in place.

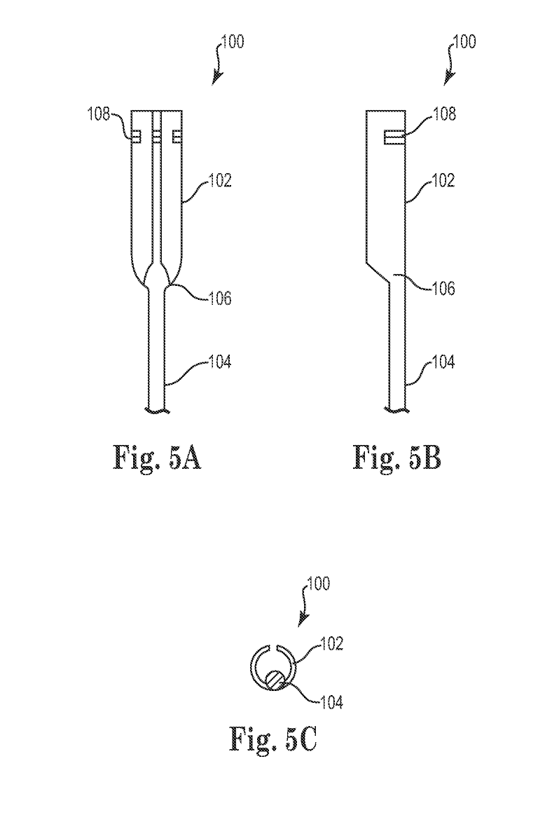

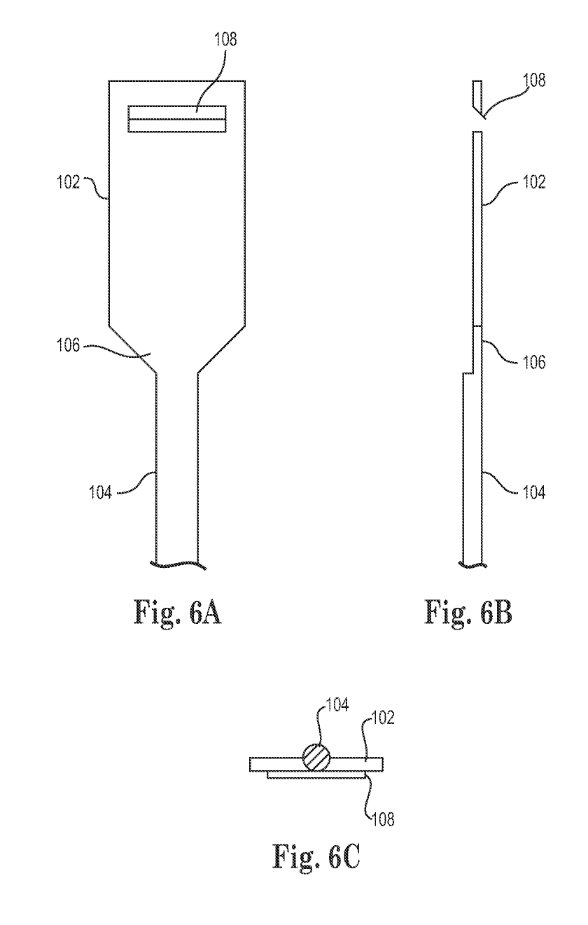

FIGS. 5A, 5B and 5C are top, side and end view illustrations, respectively, of a scoop necrosectomy assist device 100 in accordance with another embodiment of the invention. As shown, the device 100 has a scoop 102 on the tip or distal end of a catheter type handle 104. The device 100 is shown in a retracted, reduced diameter state in FIGS. 5A-5C. The device 100 is shown in its open operational state in FIGS. 6A-6C. The scoop 102 of device 100 has a generally flat structure in its neutral operational state, when no external forces are applied. The scoop 102 and optionally other portions of the device 100 can be formed from elastic or shape memory materials such as stainless steel, nitinol and polymers. The scoop 102 can be rolled or otherwise elastically deformed to the reduced-diameter retracted state. A tapered transition structure 106 facilitates the movement of the scoop 102 between the retracted and operational states in the manner described below. The illustrated embodiment of scoop 102 also has a cutting blade 108 on its distal end. Other embodiments (not shown) have a plurality of cutting blades such as 108.

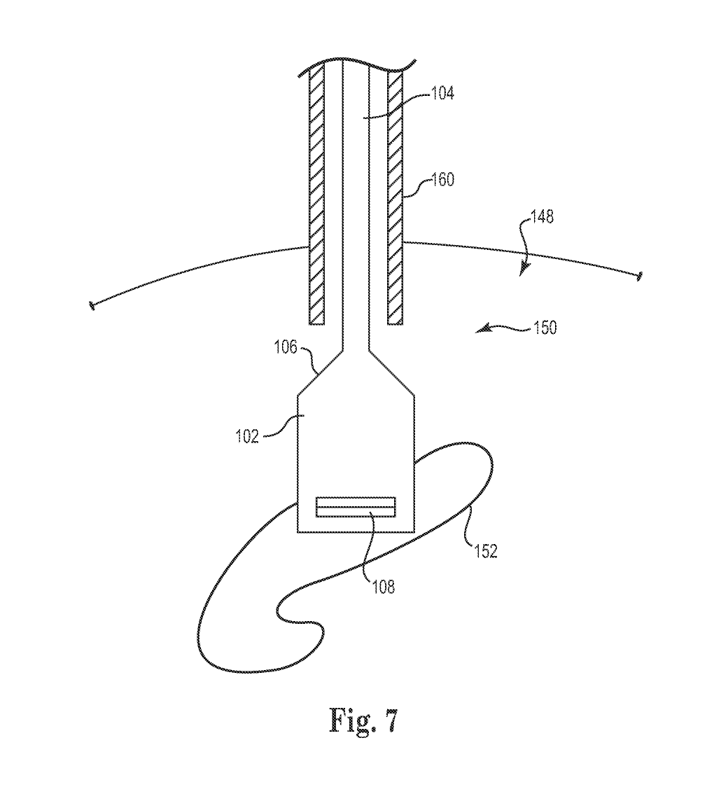

FIG. 7 is an illustration of a patient 148 undergoing a surgical procedure during which the device 100 is used to treat walled off pancreatic necrosis (WOPN). The scoop 102 can be manually positioned in its retracted state by the surgeon and positioned within the endoscope. The device 100 is inserted into the peritoneal cavity 150 through the accessory channel of an endoscope 160. As shown, when the scoop extends from the distal end of the endoscope 160 in the peritoneal cavity 150 the scoop will self-expand to its operational state. A surgeon can then manipulate the scoop 102 and blade 108 using the handle 104 to remove and collect necrotic tissue from the patent's pancreas 152. When the handle 104 is pulled to withdraw the device 100, the transition structure 106 will engage the distal end of endoscope 160 and force the scoop 102 back to its retracted position so the collected tissue can be withdrawn from the patient through the endoscope.

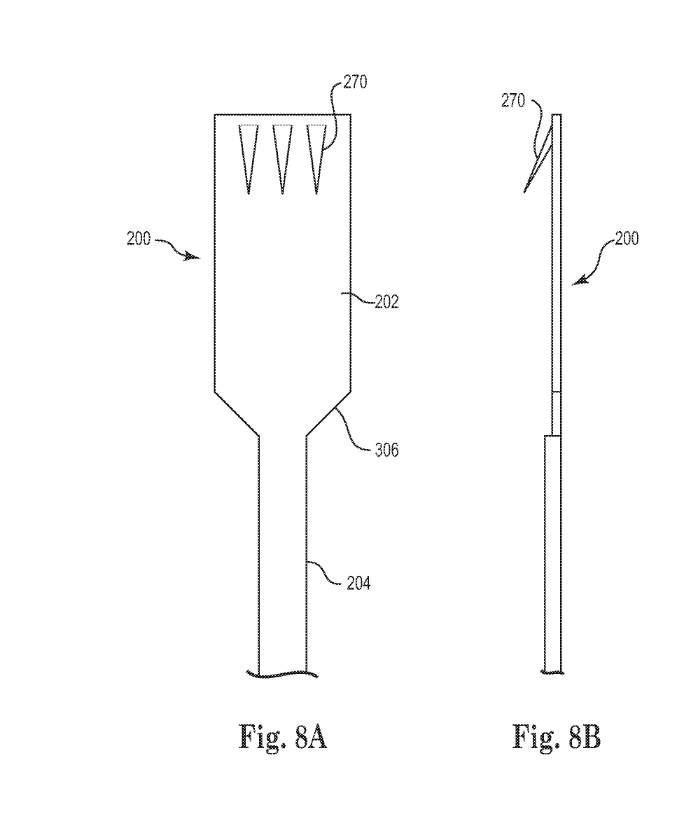

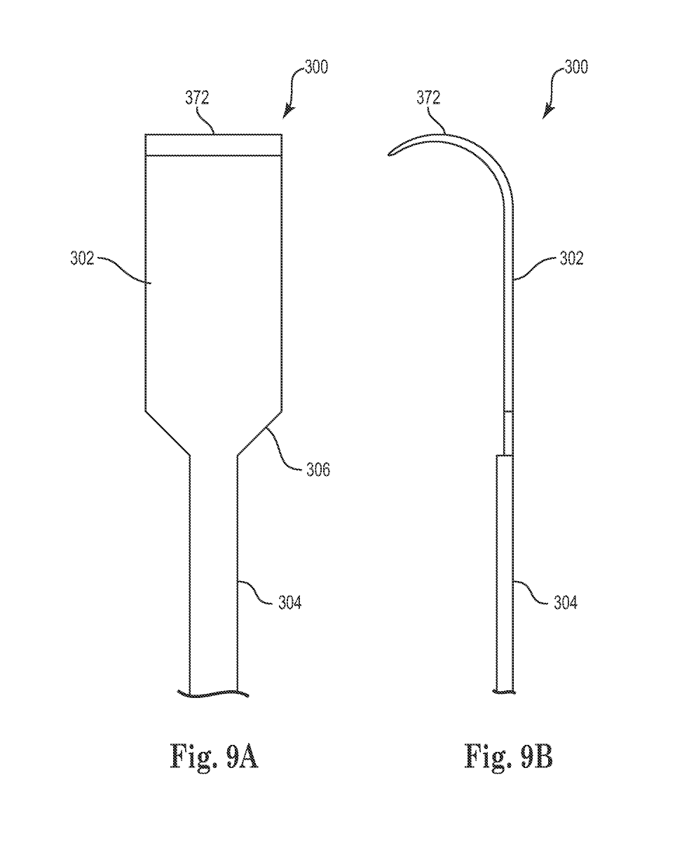

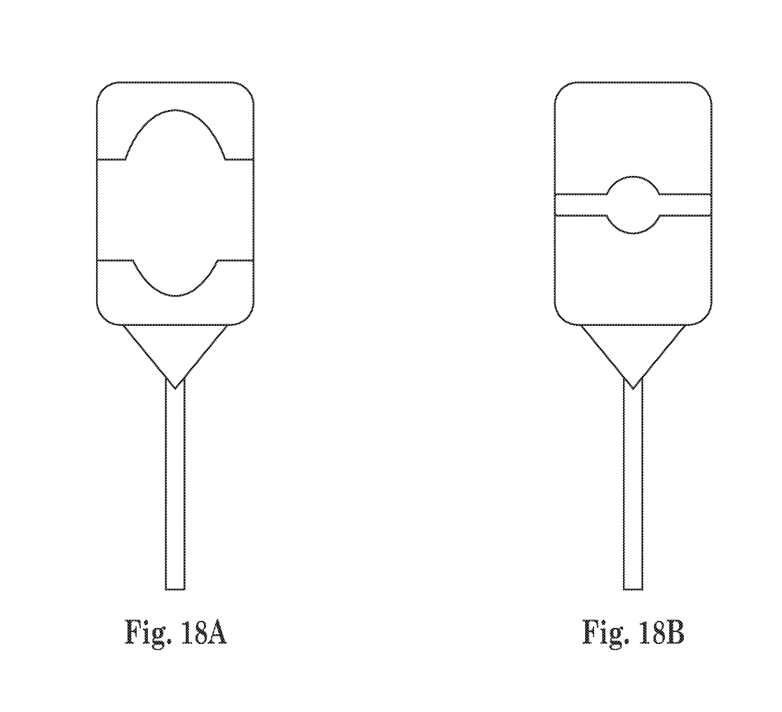

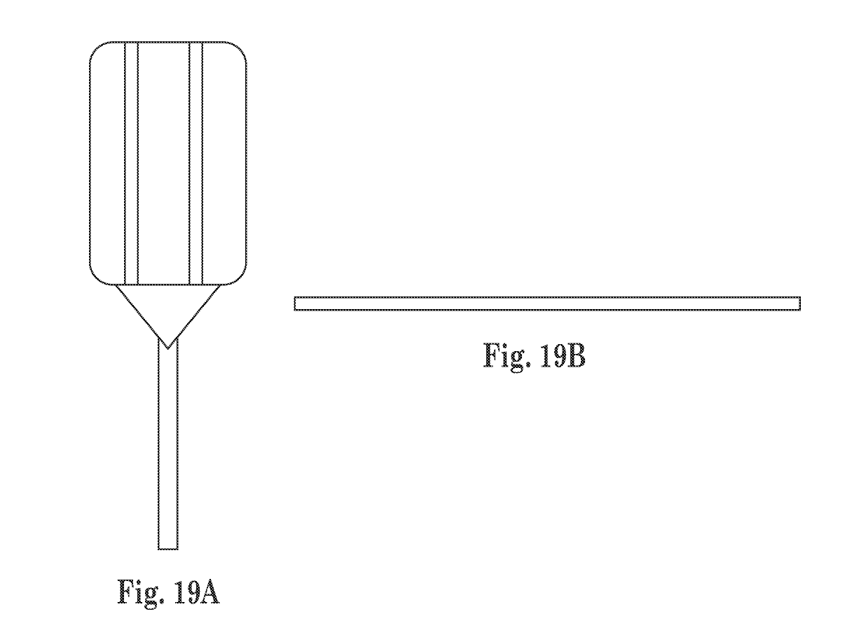

Device 100 can include other structures to move the scoop between the refracted and operational states. For example, an actuating mechanism on the handle that remains outside of the patient when the device is being used can be connected by a linkage to a scoop structure, and actuated to drive the scoop between the retracted and operational states. The scoop 102 can also be configured in such a manner that it is biased to the retracted state, with the device 100 including an actuator structure that expands the scoop to the operational state after it is inserted into the patient. The scoop can also include other structures, including other structures for enhancing the device's capability to collect tissue (e.g., fork-like extensions). FIGS. 8A and 8B, for example, illustrate an embodiment of a device 200 having a rake tip 270 with tines or prongs. FIGS. 9A and 9B illustrate an embodiment of a device 300 having a bowed tip 372. Other features of devices 200 and 300 can be similar to or substantially the same as those of device 100 described above. Still other embodiments of the invention (shown e.g., in FIGS. 18A and 18B) have reciprocating scissors or cigar cutter-type blades on the scoop that are actuated by advancing and retracting the scoop, or by an actuating mechanism. Yet another embodiment (shown e.g., in FIGS. 19A and 19B) has water infusion ports on the scoop that are coupled to a water source through lumens extending along the device. Still another version of the device (not shown) has an abrasive surface on the scoop that can be rubbed on the necrotic tissue to abrade the tissue from the pancreas. Another embodiment (not shown) includes a sphincterotome-type mechanism to enable the surgeon to bow the scoop.

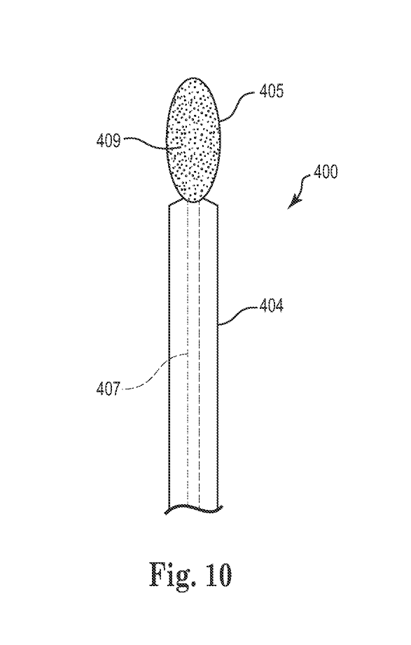

FIG. 10 is an illustration of a balloon necrosectomy device 400 in accordance with another embodiment of the invention. As shown, the device 400 includes a catheter-type handle 404 with an inflatable balloon 405 on its distal end. An inflation lumen 407 extends through the handle 404 to the balloon 405. Abrasive material 409 (e.g., a roughened surface or spines) is on the surface of the balloon 405. Balloon 405 is deflated to a reduced diameter retracted state for delivery into the peritoneal cavity of a patient (e.g., through an endoscope (not shown)). Following delivery, the balloon 405 (which is connected to a source of inflation fluid, not shown) can be inflated to an enlarged diameter operation state (not shown) and the handle 404 reciprocated back and forth to rub the surface of the balloon on the necrotic tissue of the pancreas and thereby abrade the tissue. In other embodiments (not shown) the handle 404 can be connected to a device that rotates the balloon 405 (e.g., a variable speed driver). The spinning balloon 405 in this embodiment will enhance the removal of necrotic tissue.

The invention offers a number of important advantages by enhancing endoscopic necrosectomy such as during the treatment of WOPN. Although described with reference to preferred embodiments, those skilled in the art will recognize that changes can be made in form and detail without departing from the spirit and scope of the invention.

What is claimed is:

1. A banding necrosectomy device, including:
a body;
an expandable cap on a distal end of the body; and
a tissue collecting band on the cap.

2. The banding necrosectomy device of claim 1 and further including an actuating mechanism for causing the expandable cap to move between a reduced diameter retracted state and an expanded diameter operational state.

3. The banding necrosectomy device of claim 2 and further including a band actuating mechanism coupled to the band.

4. The banding necrosectomy device of claim 1 wherein the band is a rubber band.

5. The banding necrosectomy device of claim 1 wherein the band is a snare.

6. A method for collecting necrotic pancreatic tissue using the device of claim 1, including:
inserting the device into the patient's body with the cap in a reduced diameter retracted state;
expanding the cap to an expanded diameter operational state;
collecting necrotic pancreatic tissue in the expanded diameter cap;
collapsing the cap to the retracted state; and
withdrawing the device from the patient's body.

7. The method of claim 6 wherein inserting the device includes inserting the device through an endoscope.

8. A scoop necrosectomy device, including:
a handle;
a scoop on the handle, wherein the scoop is movable between a reduced diameter retracted state and an expanded diameter operational state.

9. The scoop necrosectomy device of claim 8 wherein the scoop is formed from a shape memory material and is self biased to the operational state.

10. The necrosectomy device of claim 8 wherein the scoop has a generally flat configuration in the operation state.

11. The necrosectomy device of claim 8 wherein the scoop has a bowed tip in the operational state.

12. The necrosectomy device of claim 8 and further including one or more blades on the scoop.

13. The necrosectomy device of claim 8 and further including one or more rake tips on the scoop.

14. The necrosectomy device of claim 8 and further including an abrasive surface on the scoop.

15. The necrosectomy device of claim 8 and further including a transitional structure for causing the scoop to move from the operational state to the refracted state.

16. A method for collecting necrotic pancreatic tissue using the device of claim 8, including:
inserting the device into the patient's body with the scoop in a reduced diameter retracted state;
expanding the scoop to an expanded diameter operational state;
abrading and/or collecting necrotic pancreatic tissue through motion of the scoop;
collapsing the scoop to the retracted state; and
withdrawing the device from the patient's body.

17. The method of claim 16 wherein inserting the device includes inserting the device through an endoscope.

18. A balloon necrosectomy device, including:
a handle;
an inflatable balloon on an end of the handle; and
an abrasive surface on the balloon.

19. A method for collecting necrotic pancreatic tissue using the device of claim 18, including:
inserting the device into the patient's body with the balloon in a reduced diameter retracted state;
inflating the balloon to an expanded diameter operational state;
abrading necrotic pancreatic tissue through motion of the balloon;
deflating the balloon to the retracted state; and
withdrawing the device from the patient's body.

20. The method of claim 19 wherein inserting the device includes inserting the device through an endoscope.
